(19) United States
(12) Patent Application Publication
(10) Pub. No.: US 2007/0225812 A1
(43) Pub. Date: Sep. 27, 2007

(54) Intervertebral Prosthesis

(76) Inventor: Steven Streatfield Gill, Bristol (GB)

Correspondence Address: KENNETH I KOHN;KOHN & ASSOCIATES, PLLC, 30500 NORTHWESTERN HWY., SUITE 410, FARMINGTON HILLS, MI, 48334, US

(21) Appl. No.: 10/577,879

(22) PCT Filed: Oct. 26, 2004
(86) PCT No.: PCT/GB04/04523

§ 371 Date: Jan. 16, 2007

(30) Foreign Application Priority Data
Oct. 30, 2003 (GB) 0325421.6

(51) Int. Cl. A61F 2/44; A61B 17/00

(52) U.S. Cl. 623/17.15; 606/85

(57) ABSTRACT

The present invention relates to an intervertebral prosthesis. In particular, the present invention relates to an articulating intervertebral disc prosthesis. In use, the intervertebral prosthesis is fixed to the end plates of vertebrae following the surgical excision of a degenerative or ruptured disc. The intervertebral prosthesis has substantially flat bone engaging surfaces with a macro-textured surface.

The present invention relates to an intervertebral prosthesis. In particular, the present invention relates to an articulating intervertebral disc prosthesis. In use, the intervertebral prosthesis is fixed to the end plates of vertebrae following the surgical excision of a degenerative or ruptured disc. The present invention also relates to a method and tools for inserting and removing the intervertebral prosthesis of the present invention.

The surgical treatment of diseases of the spine commonly involves removal of the intervertebral discs. This is particularly so when there has been degeneration or rupture of the disc causing compression of the spinal cord or nerve roots resulting in neurological symptoms. Following removal of a disc it is usual surgical practice to transplant a bone graft or insert an artificial fusion element into the disc space in order to ensure the appropriate spacing and alignment of the vertebrae.

Fusion of the adjacent vertebrae that follows may however predispose neighbouring spinal motion segments to rapid deterioration. Consequently, many patients may require additional disc removal and/or fusion procedures as a result of a spinal fusion. In order to avoid the latter and to maintain spinal mobility, several different types of intervertebral disc arthroplasy devices have been proposed. These devices include attempts to mimic the cushion like properties of an intervertebral disc by replacing it with elastomeric materials, hydroscopic agents contained in a bag or a filled bladder. Alternative designs attempt to maintain vertebral separation and movement with more mechanical devices. Typically these comprise two metal plates or members that articulate such that when the device is placed in the intervertebral space, one plate engages with the end plate of the vertebra above and the other with the endplate of the vertebra below. The proposed means of articulation include hinging mechanisms allowing rotational movement in one plane; an upper plate having a convex lower surface that engages congruently with a concavity in the upper face of the lower plate that allows rotational movement in several planes; an upper plate with a convex lower surface that engages in a trough in the upper face of the lower plate that allows rotational as well as translation movement; and an ovoid polyurethane nucleus contained between opposing cavities in the plates that also allows rotational and translational movement.

Various methods of fixing artificial disc prostheses within the intervertebral space have been described. These include flat bone engaging surfaces which are porous, allowing bone ingrowths, as described in U.S. Pat. No. 3,867,728 (Stubstad et al) and U.S. Pat. No. 4,911,718 (Lee et al). It has however not been demonstrated that these devices will provide sufficient attachment to the vertebrae prior to the proposed bony ingrowth to prevent ejection of the prosthesis with consequent damage to the spinal cord or nerves.

Alternatively, the bone engaging surfaces of a prosthetic disc may be provided with protuberances, including studs, teeth, spikes, ridges or screws that penetrate the bony end plates as described in U.S. Pat. Nos. 5,701,437 (Stefee); 4,759,766 (Buettner-Janz et al); 4,309,777 (Patil); 5,123,926 (Pisharodi); 4,874,389 (Downey). To implant such devices the adjacent vertebrae must be spread substantially further apart than their normal distance from each other so that the prosthesis can be maneuvered between the vertebrae and fixed in place. This operation presents additional risk of injury to the vertebrae and soft tissues, which may lead to heterotopic calcification in ligaments and ultimately to fusion of the motion segment.

A cervical disc prosthesis described by Bryan, U.S. Pat. Nos. 5,674,299, 5,865,846, 6,001,130 and 6156067 has convex metallic bone engaging surfaces that are porous to encourage bony ingrowth. To insert the device the opposing articular faces of the vertebra to which it will be attached are exactly machined with concavities to accept and retain it. This requires a complexity of instrumentation that necessitates prolonged operating time and increased tissue trauma. Insertion of the device also requires over-distraction of the adjacent vertebrae with the consequences as described above.

U.S. Pat. No. 6,113,637 (Gill et al) describes a method of fixing a disc prosthesis to adjacent vertebrae using screws inserted through anterior flanges on each joint articulation. In order to insert the joint into the disc space, geometrically precise preparation of the anterior faces of the vertebrae relative to the articular faces is critical if stable fixation of the joint is to be achieved. This may be problematic if the anterior face of the vertebra becomes deformed from excessive osteophyte formation resulting in, for example, a wedge shaped or concave anterior face. Correction of such deformity may require excessive removal of cortical bone with subsequent weakening of screw fixation. Failure to adequately prepare the geometric relationship between the anterior and articular faces of the vertebrae prior to joint insertion may lead to rocking of the joint fixation and excessive stress being placed on the screws that may loosen or fracture.

For long term stability of the prosthetic disc it is desirable that the bone engaging surfaces are porous or textured and allow bone ingrowth, and that the area of engagement of this surface with the bony end plates is maximised. Various texturing methods have been developed to treat the surfaces of metallic implants to improve their biological fixation strength. These include the application of small spherical particles to the surface that are made of the same metal as the implant. These particles can be applied to the bone engaging surface of the implant by vapour deposition, by plasma jet spraying or by any other suitable known technique. The surfaces of implants may also be mechanically or chemically etched or textured. Chemical texturing methods are available commercially, for example; Chem Tex® 5-5-5 (CYCAM inc; Houston, Pa.) and Tecotex® 1-103 (Tecomet, Woburn, Mass.). The latter texturing method produces a coarse pocketed surface with the radius of the pockets typically greater than 0.4 mm; otherwise known as a macro-textured surface. Macro-textured surfaces have potential advantages over less coarse or bead coated surfaces in biological fixation because they will more readily embed themselves into bone and during insertion may rasp the bone surface shaving off small particles that are forced into the recesses of the texture and thereby encourage bone growth into the surface. When bone has grown into a macro-textured surface the sheer strength is high and should be more than sufficient to retain the prosthesis in situ.

It is sometimes desirable to remove a disc prosthesis due to poor initial placement or subsidence of the prosthesis into the endplates of the vertebra, causing malarticulation and pain, or as a result of facet joint pain provoked by re-establishing movement at a previously diseased and relatively immobile segment. A disc prosthesis may also need to be removed if it becomes infected or if there is recurrent growth of osteophytes at the same level causing myelopathy or radiculopathy. If the implanted disc prosthesis has a bone ingrowth surface then its removal could be achieved by drilling bone from around it to free it, but this will lead to significant bone loss making subsequent spinal fusion or replacement with another prosthetic disc very difficult. Separation of the prosthesis from the vertebral end plates is preferably carried out using an osteotome or chisel.

However, this demands that the textured surface allowing bony ingrowth is applied to a flat surface and devices which have protruberances arising from a generally flat bone engaging surface, as described in the prior art, will obstruct the passage of a chisel or osteotome making disengagement extremely difficult. Similarly if the bone engaging surface is dome shaped, as described in U.S. Pat. No. 5,865,846, the surgeon would face similar difficulties. The use of a bone ingrowth surface would not be recommended when the prosthesis employs relatively large anterior flanges for screw fixation, as described in U.S. Pat. No. 6,113,637, because they will obstruct access to the surface to facilitate its disengagement from the bone.

Therefore there remains a need for an intervertebral prosthesis that is easy to implant in an intervertebral space such as a disc space, without over distraction of the vertebrae. The surface of the prosthesis in contact with the bone should have a surface that will provide secure initial fixation and allow bone ingrowth to secure long-term stability. The disc prosthesis should be designed in such a way that its subsequent removal can be accomplished safely without significant bone destruction or loss. The prosthesis is preferably provided with a placement tool that will enable its implantation and removal.

The present invention is directed to a simple intervertebral prosthesis that can be fixed to vertebral end plates following the removal of a diseased, ruptured or degenerative disc.

The present invention provides an intervertebral prosthesis comprising a first plate and a second plate, wherein each plate comprises a substantially flat bone engaging surface with a macro-textured surface capable of rasping an interstitial bone surface, and one or more lugs which on intervertebral insertion of the prosthesis abut a non-interstitial surface of the vertebrae to limit insertion of the prosthesis.

The substantially flat bone engaging surfaces provide a coarse rasp-like surface that can be mechanically embedded into the bone after it is placed between vertebrae by moving the prosthesis so that the bone engaging surfaces rasp against the interstitial bone surfaces, namely the vertebral end plates. Preferably a to-and-fro rotational movement is applied to the prosthesis. Mechanical engagement of the macro-textured surfaces into the vertebral end plates provides a primary fixation of the prosthesis. The primary fixation is sufficient in order to hold the intervertebral prosthesis in position. After primary fixation, bone ingrowth into the recesses of the bone engaging surfaces provides long-term stability.

The macro-textured bone engaging surfaces form a sufficiently strong bond with the vertebral end plates so that the intervertebral prosthesis does not need to be fixed in place by any fixation means, such as screws or the like, despite the large amount of intervertebral movement that occurs. The intervertebral prosthesis is held in position because the main force acting on the prosthesis is compression between the vertebrae. When the plates are articulated together to allow a physiological range of movement between the plates any other forces acting on the plates will be dissipated by the articulation. This results in the compressive force being substantially the only force acting on the prosthesis.

Preferably the macro-textured surfaces are disc shaped. Disc shaped macro-textured surfaces assist in allowing the prosthesis to be rotated when inserted between vertebrae. The prosthesis is rotated to embed it into the vertebral end plates.

Preferably, the intervertebral prosthesis of the present invention does not comprise any fixing means, such as screws, etc., or any holes for receiving such fixing means enabling it to be fixed to a non-interstitial surface of a vertebra.

Preferably, each plate of the intervertebral prosthesis comprises a non-textured area located between the macro-textured surface and the one or more lugs. The purpose of non-textured area is so that when the intervertebral prosthesis is inserted and the macro-textured surface rasps and penetrates the vertebral end plates, the area of bone which is contacted by the non-textured area is preserved, namely the bone does not form a bond with the non-textured area. The presence of the non-textured area helps to retain the prosthesis within the intervertebral space and also assists in any subsequent removal of the intervertebral prosthesis. It is particularly preferred that the non-textured area is a recessed area and is preferably approximately 0.4 to 1 mm lower than the macro-textured surface. It is also preferred that the non-textured area forms a border between the macro-textured surface and the one or more lugs and wherein the border may have a variable width of between 2 and 10 mm.

The term “non-textured area” means a surface area that has not been macro-textured and an area on which bone ingrowth cannot occur. The non-textured area may be flat and smooth or may be shaped so that bone can contact the surface and possible engage the surface, but not bond with the surface.

The presence of the non-textured area ensures that the face of the bone which engages the non-textured surface acts as a buttress and helps retain the prosthesis within the intervertebral space.

The one or more lugs of the intervertebral prosthesis prevent movement of the prosthesis by acting as stops which abut a non-interstitial surface of the vertebrae. Preferably each plate comprises two lugs. It is further preferred that the lugs are positioned on one side of the intervertebral prosthesis. In use, the intervertebral prosthesis of the present invention is generally inserted between the vertebrae in an anterior to posterior direction. The leading edge of the intervertebral prosthesis, namely the edge that is inserted into the intervertebral gap is termed the posterior edge. The opposing edge is termed the anterior edge. As will be appreciated, the one or more lugs are preferably positioned on the anterior edge in order to prevent further posterior displacement of the prosthesis by abutting the non-interstitial surface of the vertebrae.

The lugs are preferably mounted on each end of the anterior edge of the first and second plates. It is particularly preferred that the lugs are capable of engaging an insertion tool enabling the plates to be manipulated using the insertion tool. In a particularly preferred embodiment, the lugs are prism shaped. Prism shaped lugs can withstand large forces and are particularly suitable for connection to a holding tool, which is discussed further below. Preferably, each prism shaped lug is orientated so that its flat base faces anteriorly and the posteriorly facing apical edge will engage with the anterior, non-interstitial surfaces of the vertebrae.

It should be noted that the lugs abut a non-interstitial surface of the vertebrae but are not fixed to the vertebrae using a fixing means. By avoiding having to fix the intervertebral prosthesis to the vertebrae, the insertion procedure is simplified and quicker. Furthermore, less damage is caused to the vertebrae as screws or other fixing means do not have to be inserted.

The first and second plates are articulated together so as to provide a physiological range of motion between the plates. Numerous constructs for allowing suitable movement between the plates are known to those skilled in the art.

The bone engaging surfaces of the intervertebral prosthesis of the present invention are substantially flat. This means that the bone engaging surfaces do not comprise a series of large peaks and troughs but instead that the macro-textured surface is sufficiently uniform so as to provide a rough surface which is still substantially flat so that the bone engaging surface can be separated from vertebral end plates by using an osteotome or chisel. By avoiding large peaks and troughs on the bone engaging surface, the bone engaging surface can be removed from the vertebral end plates by simply cutting across the substantially flat surface of the bone engaging surface. Such a method has the advantage that only a small amount of bone is lost on removing the intervertebral prosthesis.

The macro-textured surface can be produced by any known method including mechanical or chemical methods. Chemical texturing methods include Chem Tex® 5-5-5 (CYCAM Inc, Houston, Pa.) and Tecotex® 1-103 (Tecomet, Woburn, Mass.). The Surfi-Sculp™ technique (TIWI Limited, Cambridge, UK) may be used to produce the macro-textured surface. The Surfi-Sculpt™ technique comprises using electron beams to re-shape materials precisely. It is essential that the macro-textured surface is sufficiently coarse so as to rasp an interstitial bone surface. In particular, the macro-textured surface must on insertion or when moved (e.g. rotated) after insertion, rasp the bone surface and thereby shave off small particles that are forced into the recesses of the macro-textured surface and thereby encourage bone growth into the surface. Preferably, the macro-textured surface comprises a number of pockets or pits having a radius of generally greater than 0.4 mm. Suitable macro-textured surfaces are well known to those skilled in the art.

Preferably, the articulation between the end plates should be weight bearing, maintain normal separation between the articulating vertebrae and facilitate a physiological range of motion between the vertebrae. It is particularly preferred that the first and second end plates are articulated together via the mechanism described in U.S. Pat. No. 6,113,637 (Gill et al).

The intervertebral prosthesis can be made from any biocompatible material. Suitable materials include stainless steel, titanium, titanium carbide, zirconium or their equivalents.

It is also preferred that there is a chamfer at the posterior edge of each plate of the intervertebral prosthesis. The chamfers assists with the insertion of the intervertebral prosthesis.

The present invention also provides an abrasive trial device having substantially the same dimensions as the intervertebral prosthesis of the present invention, and includes one or more lugs that correspond in size, shape and position to those of the intervertebral prosthesis of the present invention, wherein the surfaces of the plates of the trial device have an abrasive surface for smoothing the vertebral end plates. On intervertebral insertion of the abrasive trial device the one or more lugs abut a non-interstitial surface of the vertebrae to limit insertion of the abrasive trial device.

The abrasive trial device is used in sizing and preparing the intervertebral space so that it can accept the intervertebral prosthesis of the present invention. The abrasive trial device has substantially the same dimensions as the intervertebral prosthesis. In other words, the abrasive trial device is substantially the same shape and size as the intervertebral prosthesis except that it is lower than the prosthesis by the combined height of the macro-texturing of the first and second plates. It is envisaged that various sizes of the intervertebral prosthesis of the present invention would be available for the surgeon to implant, and similarly various abrasive trial devices would be provided to match the profile of the intervertebral prosthesis.

The one or more lugs on the abrasive trial device act in the same manner as the lugs on the intervertebral prosthesis. In particular, the lugs act to prevent over insertion of the abrasive trial device.

The plate surfaces of the abrasive trial device contact the vertebral end plates and are provided with an abrasive surface enabling the vertebral end plates to be smoothed so as to prepare the intervertebral space to accept the invertebral prosthesis. The abrasive surfaces can be obtained by providing a series of cutting edges or a diamond studded surface, to produce a file or fine rasp-like surface.

In particular, the abrasive trial devices are used in sizing and preparing the invertebral space so that it can accept the invertebral prosthesis of the present invention.

The abrasive trial device can be connected to a insertion tool in the same manner as the invertebral prosthesis, namely via the one or more lugs. Alternatively, the abrasive trial device can be formed as a single unit with an elongated handle enabling its insertion into an intervertebral space.

As indicated above with, respect of the invertebral prosthesis of the present invention, the one or more lugs are preferably two prism shaped lugs located on the anterior edge of the invertebral prosthesis. Preferably, the abrasive trial device comprises identical lugs at the same position.

Preferably, each prism shaped lug is orientated so that it flat base faces anteriorly and the posteriorly facing apical edge will engage with the anterior, non-interstitial surfaces of the vertebrae. The advantage of this arrangement is that if the abrasive trial device is tapped home within the intervertebral space, the lugs will indent the anterior vertebral non-interstitial surface thereby marking their points of contact. If all four lugs fail to make proper contact with the vertebrae, then the surgeon can remove any bone irregularities to ensure that the invertebral prosthesis will seat properly. Of course, the process of impacting the lugs into the anterior vertebral bodies should create complementary recesses will also assist in this manner.

Trial devices that do not have abrasive surfaces, herein referred to has sizing trial devices, can be used to check the size of the intervertebral space. The sizing trial device will be of the same shape and size as the prosthesis to be inserted except that it is lower than the prosthesis by the combined height of the macro-texturing of the first and second plates.

The present invention also provides a sizing trial device having substantially the same dimensions as the intervertebral prosthesis of the present invention, and includes one or more prism shaped lugs that correspond in size, shape and position to those of the intervertebral prosthesis of the present invention.

The present invention also provides an insertion tool which can be releasibly attached to the invertebral prosthesis of the present invention. In particular, the present invention provides an insertion tool comprising:
a shaft having a proximal end and a distal end wherein the proximal end comprises a grip and the distal end comprises two prosthesis engaging arms that are biased apart; and
a cylinder having a proximal and distal end in rotational engagement around the main shaft,
   wherein the distal end of the cylinder contacts the prosthesis engaging arms, and wherein on rotation of the cylinder in a distal direction, the distal end of the cylinder forces the prosthesis engaging arms together to grip an invertebral prosthesis according to the present invention.

In use, the surgeon will grip the proximal end of the shaft and rotate the cylinder so that it moves in a distal direction. As the cylinder moves in a distal direction, it will engage the prosthesis engaging arms and force them together due to the distal movement of the cylinder. As the prosthesis engaging arms are forced together, the arms can grip the invertebral prosthesis according to the present invention. Release of the invertebral prosthesis can be achieved by simply rotating the cylinder to cause it to move in a proximal direction.

Preferably, the cylinder's proximal end is threaded internally and engages with a correspondingly threaded section of the shaft.

The prosthesis engaging arms are shaped so that on distal movement of the cylinder, the arms are forced together. Preferably, the prosthesis engaging arms comprise recesses to engage with the one or more lugs formed on each plate of the invertebral prosthesis of the present invention. The recesses are formed so that the invertebral prosthesis can be firmly gripped when the two arms are forced together. Preferably, the invertebral prosthesis of the present invention comprises one or more, most preferably two, prism shaped lugs on each plate. It is therefore preferred that the two prosthesis engaging arms of the insertion tool comprise complementary recesses in the engaging arms to enable the invertebral prosthesis to be firmly gripped by the insertion tool. The use of prism shaped lugs and reciprocal recesses in the arms of the holding tool is particularly advantageous as a firm grip is achieved. The firm grip is achieved because each prism shaped lug of the prosthesis is held on four faces by each recess in the arms of the tool. Furthermore, due to the reciprocal shape of the prism-shaped lugs and recesses, the lugs are self centering on connection with the holding tool. The position of the lugs within the recesses is constrained and the first and second plates are locked in position relative to one another. Furthermore, the arms of the holding tool only need to move a short distance in order to switch between a gripping position and a non-gripping position.

The trial devices and insertion tool of the present invention may be made of any suitable biocompatible material such as stainless steel, titanium, titanium carbide, zirconium or their equivalents.

The present invention also provides a kit comprising one or more invertebral prostheses according to the present invention, one or more abrasive trial devices according to the present invention and one or more sizing trial devices according to the present invention.

Preferably the kit also comprises an insertion tool according to the present invention.

Preferably, the kit comprises a series of different sized invertebral prosthesis as well as a corresponding series of different sized abrasive trial devices and sizing trial devices.

The present invention also provides a method for inserting the intervertebral prosthesis of the present invention comprising:
preparing the vertebral end plates between which the invertebral prosthesis is to be inserted, by inserting and moving the abrasive trial device of the present invention within the invertebral space so as to smooth the end plates and to ensure that the one or more lugs of the trial device engage a non-interstitial surface of the vertebrae; and
inserting the intervertebral prosthesis into the prepared intervertebral space; and
moving the prosthesis within the intervertebral space to ensure that the macro-textured surface engages the vertebral end plates.

The method may comprise an additional step of checking the size of the intervertebral space using the sizing trial device of the present invention prior to insertion of the prosthesis. As will be appreciated by those skilled in the art, the insertion and movement of both the trial device and intervertebral prosthesis can be achieved by using the insertion tool of the present invention.

When the macro-textured surfaces of the invertebral prosthesis have been mechanically engaged into the end plates of the vertebrae, preferably by lateral rotation of the plates within the intervertebral space, the prosthesis will be sufficiently stable to facilitate its normal and immediate functioning. The insertion process will have seeded the recesses of the macro-textured surface with fine bone particles, which will stimulate bone ingrowth and so provide permanent stability.

The present invention also provides a method for removing the invertebral prosthesis of the present invention after bone ingrowth into the macro-textured surfaces. Accordingly, the present invention provides a method for removing the invertebral prosthesis of the present invention from an intervertebral space comprising passing a fine chisel or osteotome between the macro-textured surface of each end plate and the vertebral end plates.

The fine chisel or osteotome can be provided with a stop in order to restrict the amount that it can be inserted. As will be apparent to those skilled in the art, the holding tool of the present invention can be used to withdraw the prosthesis by engaging the lugs of the prosthesis.

The present invention is now described by way of example only with reference to the following figures.

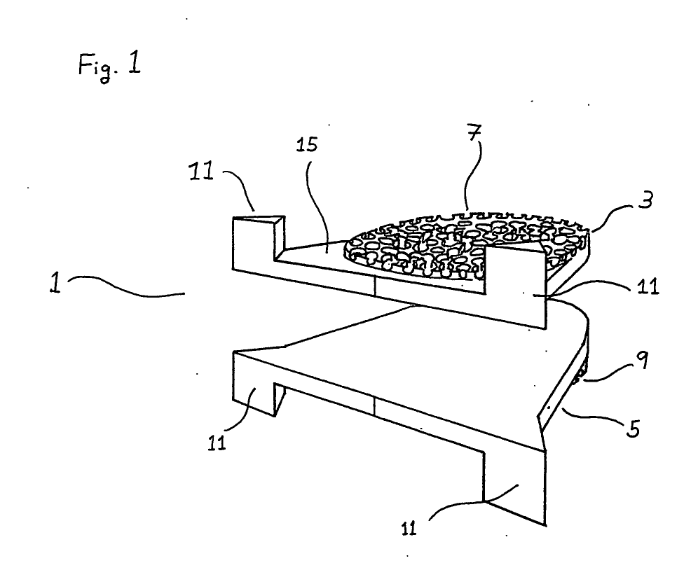

FIG. 1 shows a perspective view of the upper and lower plates of the intervertebral prosthesis (the articulation that it positioned between the plates is not shown).

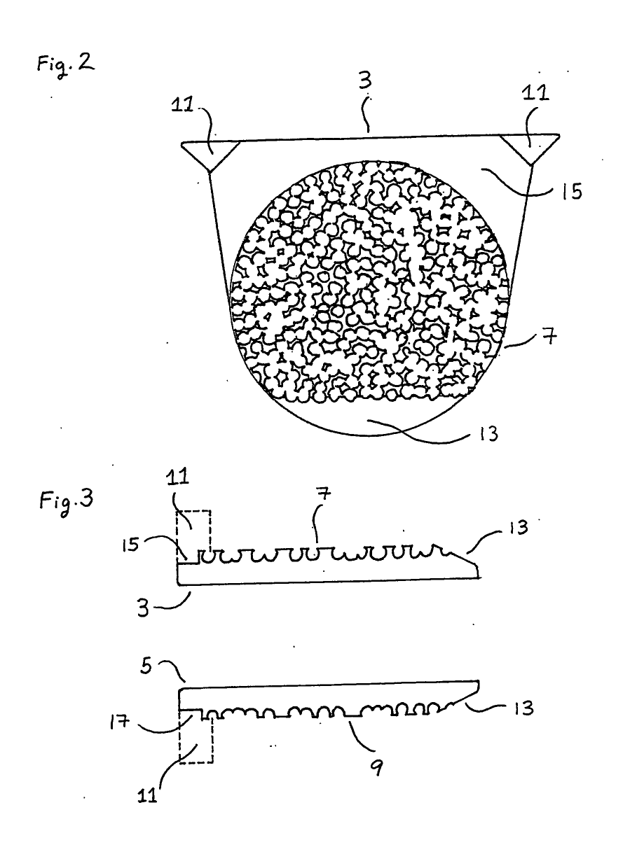

FIG. 2 shows a superior view of the upper plate of the intervertebral prosthesis.

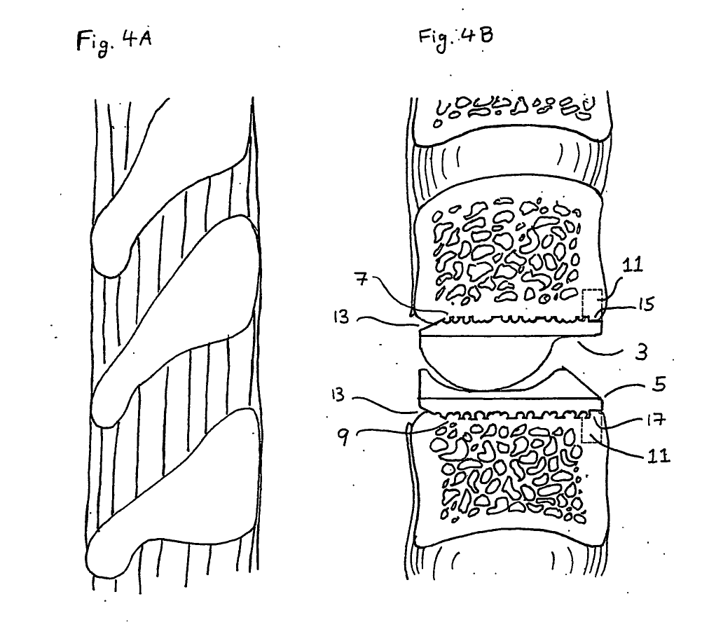

FIG. 3 shows a cross sectional lateral view of both the upper plate and lower plate of the intervertebral prosthesis.

FIG. 4 shows a cross sectional view through a spine (FIG. 4a) wherein the intervertebral prosthesis has been inserted (FIG. 4b) wherein, by way of example only, the articulation shown between the first and second end plates is a ball and trough type such as described in U.S. Pat. No. 6,113,637.

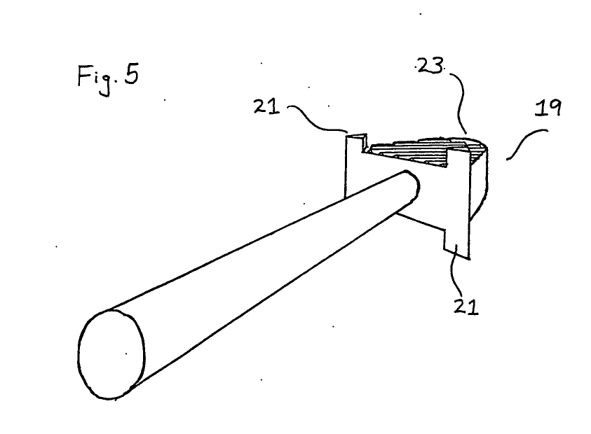

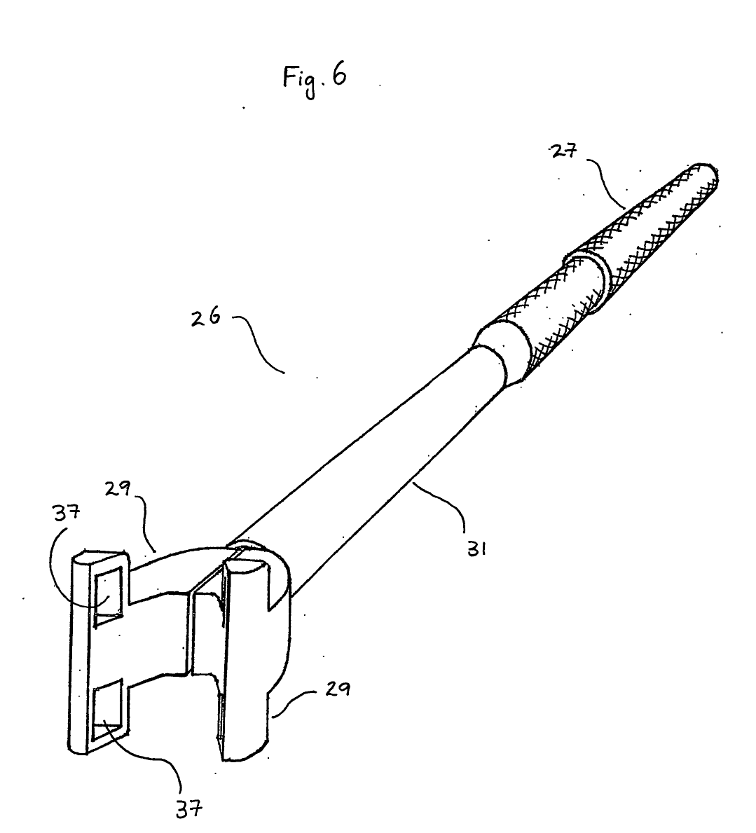

FIG. 5 shows a perspective view of an abrasive trial device according to the present invention.

FIG. 6 shows a perspective view of the holding tool according to the present invention.

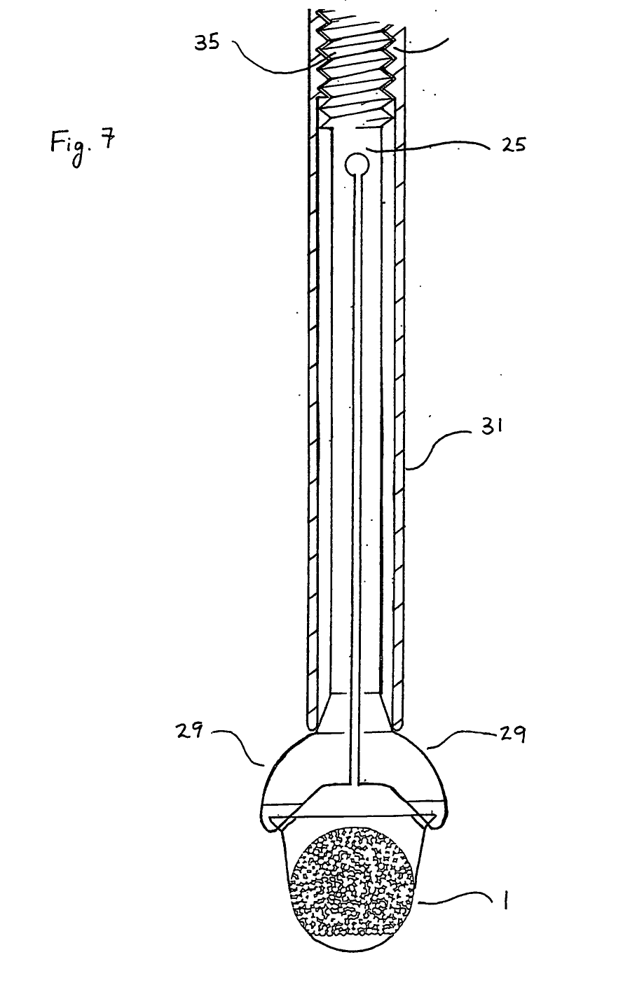

FIG. 7 shows a cross sectional view through the holding tool of the present invention.

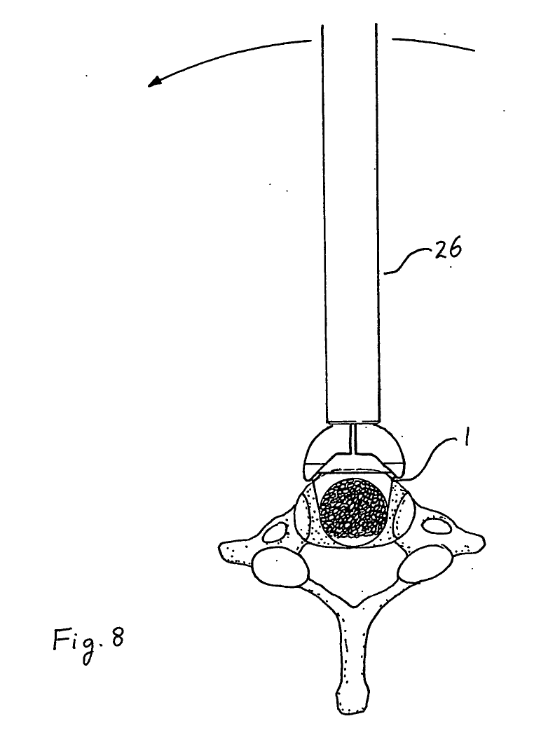

FIG. 8 shows the insertion of the intervertebral prosthesis into a cervical disc space illustrating the to-and-fro rotatary movement that imbeds the macro-textured surface of the intervertebral prosthesis into the vertebral end plates.

### EXAMPLES

### Example 1

### The Prosthesis

An intervertebral prosthesis (1) is shown in FIG. 1 having a first plate (3) and a second plate (5). The intervertebral prosthesis is manufactured from titanium carbide. The plates (3, 5) are articulated together using any suitable mechanism. In the prosthesis shown in FIG. 4b, the ball and trough articulation described in U.S. Pat. No. 6,113,637 is shown. Each plate (3, 5) has a bone engaging surface (7, 9) with a macro-textured surface. The macro-textured surface is produced by chemical texturing methods such as Chem Tex® or Tecotex® and comprises a pocketed surface where the radius of the pockets is typically greater than 0.4 mm. The macro-textured surface is substantially flat. The height of the pockets formed in the macro-textured surface are substantially the same so that a substantially uniform macro-textured surface is formed.

Each plate (3, 4) has a chamfered posterior edge (13) as shown in FIGS. 2 and 3. The posterior edge (13) is the leading edge of the prosthesis (1), namely the edge that is inserted into the intervertebral gap. The opposing edge is the anterior edge. The chamfered posterior edge (13) does not have a macro-textured surface and assists with insertion of the prosthesis (1).

Between the anterior edge of the plates (3, 5) and the macro-textured surface (7, 9), there is a non-textured recessed border (15, 17). After the macro-textured surface (7, 9) has been embedded in the vertebral end plates, the non-textured recessed border (15, 17) will engage a buttress of bone at the anterior edge of the vertebrae which has been preserved i.e. the bone has not been rasped by the macro-textured surface, and thereby help retain the prosthesis (1) within the intervertebral space.

The prosthesis (1) comprises prism shaped lugs (11) on each end of the anterior edge of the plates (3, 5). The flat base of the prism shaped lugs (11) is aligned with the anterior edge of the plates (3, 5) and the apical edge of the prism shaped lugs is positioned so that it will abut a non-interstitial surface of the vertebrae when the prosthesis is inserted.

In FIG. 4b the prosthesis (1) of the present invention is shown within an intervertebral space. The macro-textured surfaces (7, 9) of the plates (3, 5) have been embedded into the vertebral end plates by rotational movement of the prosthesis (1). The prism shaped lugs (11) abut the anterior surface of the vertebrae preventing any posterior movement of the prosthesis (1). A buttress of bone at the anterior edge of the vertebrae can be seen to engage the borders (15, 17) thereby helping to retain the prosthesis (1) in position.

### Example 2

### Use of the Prosthesis

An anaesthetised patent is placed supine on the operating table with the neck held in a neutral position and cervical traction applied using for example, a Holter traction system. The anterior cervical spine is exposed at the appropriate level through a transverse cervical incision.

The cervical discectomy and decompression is performed in a conventional way and the vertebral end plates then made parallel using a burr. Cervical traction is now released.

The depth and height of the disc space is then determined using a selection of abrasive trial devices. A suitable abrasive trial device (19) is shown in FIG. 5. The abrasive trial device has substantially the same dimensions as the prosthesis (1) to be fitted except is lower than the prosthesis by the combined height of the macro-texturing of the first and second plates (7, 9), and has prism shaped lugs (21) located at the corresponding position to the lugs (11) on the prosthesis (1). The abrasive trial device has end plate engaging surfaces (23) which an abrasive surface enabling it to smooth the vertebral end plates between which the prosthesis (1) is to be inserted. The abrasive surface is formed by a series of fine cutting edges. The abrasive trial device is connected to a handle enabling it to be inserted into an intervertebral space.

Final preparation of the end plates may be achieved using the abrasive trial device (19) with end plate engaging surfaces (23) that are provided with fine cutting edges. When such a device (19) is moved to and fro and in and out of the disc space it will smooth the end plate in readiness to accept an appropriately sized prosthesis (1). The abrasive trial device (19) is also used to ensure that the prosthesis (1) will be seated correctly in the disc space so that lugs present on the anterior end of both end plate engaging surfaces (23) of the abrasive trial device (19) will engage with the anterior faces of the vertebral bodies on each side of the prepared disc space. This may require the removal of additional bone.

A sizing trial device, which does not have the abrasive surfaces is selected that fits snugly in the prepared disc space but without distracting it. This sizing trial device is matched with a disc prosthesis (1) that is higher than it by an amount that is equivalent to the combined thickness of the texturing on the plates (7, 9) of the prosthesis (1). Typically this would be in the range of 0.8-2 mm.

The selected prosthesis (1) is fixed in an insertion tool (26). FIGS. 6 and 7 show a suitable holding tool (26). The tool comprises a shaft (25) having a proximal end and a distal end. A grip (27) is provided at the proximal end and a pair of prosthesis engaging arms (29) provided at the distal end. The distal end of the shaft (25) is split along its longitudinal axis to create the arms (29). The arms are formed so that they are biased apart. The tool (26) also comprises a cylinder (31) in rotational engagement around the shaft (25) wherein the distal end of the cylinder (31) engages with the divergent arms (29) present at the distal end of the shaft (25). The cylinder (31) comprises an internally threaded portion (33) that engages a threaded portion (35) of the shaft (25). Rotation of the cylinder (31) in a clockwise direction cases the causes the cylinder (31) to move in the distal direction with respect to the shaft (25) and causes the arms (29) to approximate. Rotation in the anticlockwise direction causes the arms to separate.

The arms (29) comprise lug receiving recesses (37) positioned to engage with the lugs (11) present on the prosthesis (1). When the arms (29) of the tool (26) are approximated, they firmly grip the first and second plates (3, 5) of the prosthesis (1) and fix them in an orientation suitable for implantation. This is facilitated by the prism shaped lugs (11) which are forcibly retained on four sides within their complementary recesses in the arms (29) of the holding tool (26).

Cervical traction is applied to widen the disc space and the prosthesis (1) is inserted into it. Cervical traction is released and the prosthesis (1) rotated to-and-fro a few times to ensure that the rasp like macro-textured surface (7, 9) of the prosthesis (1) engages in the vertebral end plates. Such movement is shown in FIG. 8.

The insertion tool (26) is disengaged from the prosthesis (1) and the wound is closed.

When the prosthesis macro-textured surfaces (7, 9) have been mechanically engaged into the end plates of the vertebrae, the prosthesis (1) should be sufficiently stabilised to facilitate its normal and immediate functioning. The insertion process will have seeded the recesses of the macro-textured surface (7, 9) with fine bone particles which will stimulate bony ingrowth and so provide permanent stability.

Removal of the prosthesis after bone has grown into its textured surface (7, 9) may be achieved as follows:

The appropriate motion segment is surgically exposed and the lugs (11) on the first and/or second plate (3, 5) of the prosthesis (1) held in a holding tool (26). A fine chisel or osteotome that is provided with a stop to prevent its penetration beyond the known depth of the prosthesis (1) is then passed between the lugs (11) and over the superior and/or inferior surface disengaging it from the end plate. The holding tool (26) can now be used to withdraw the prosthesis (10) from the intervertebral space.

All documents cited above are incorporated herein by reference.

What is claimed:

1. An intervertebral prosthesis comprising a first plate and a second plate, wherein each plate comprises a substantially flat bone engaging surface with a macro-textured surface capable of rasping an interstitial bone surface, and one or more lugs which on intervertebral insertion of the prosthesis abut a non-interstitial surface of the vertebrae to limit insertion of the prosthesis.

2. The intervertebral prosthesis of claim 1, wherein the macro-textured surfaces are disc shaped.

3. The intervertebral prosthesis of claim 1 that does not comprise any fixing means or holes for receiving such fixing means for fixing the prosthesis to a non-interstitial surface of a vertebra.

4. The intervertebral prosthesis of claim 1, wherein each plate comprises a non-textured area located between the macro-textured surface and the one or more lugs.

5. The intervertebral prosthesis of claim 1, wherein the non-textured area is approximately 0.4 to 1 mm lower than the macro-textured surface.

6. The intervertebral prosthesis of claim 1, wherein the non-textured area forms a border between the macro-textured surface and the one or more lugs having a variable width of between 2 and 10 mm.

7. The intervertebral prosthesis of claim 1, wherein the one or more lugs are positioned on one side of each plate.

8. The intervertebral prosthesis of claim 1, wherein each plate comprises two lugs.

9. The intervertebral prosthesis of claim 1, wherein the edge of each plate that is inserted into the intervertebral gap is termed the posterior edge and the opposing edge is termed the anterior edge, and wherein the lugs are positioned on the anterior edge of each plate.

10. The intervertebral prosthesis of claim 9, wherein there is a chamfer at the posterior edge of each plate of the intervertebral prosthesis to assists with the insertion of the intervertebral prosthesis.

11. The intervertebral prosthesis of claim 1, wherein the lugs are capable of engaging an insertion tool.

12. The intervertebral prosthesis of claim 1, wherein the lugs are prism shaped.

13. The intervertebral prosthesis of claim 12, wherein the each prism shaped lug is orientated so that its flat base faces anteriorly and the posteriorly facing apical edge will engage with the anterior non-interstitial surfaces of the vertebrae on intervertebral insertion.

14. The intervertebral prosthesis of claim 1, wherein the first and second plates are articulated together so as to provide a physiological range of motion between the plates.

15. The intervertebral prosthesis of claim 1, wherein the macro-textured surface comprises pockets or pits having a radius of generally greater than 0.4 mm.

16. The intervertebral prosthesis of claim 1 made from stainless steel, titanium, titanium carbide, zirconium or any biocompatible equivalent.

17. An abrasive trial device having substantially the same dimensions as the intervertebral prosthesis according to claim 1, and including one or more lugs that correspond in size, shape and position to those of the intervertebral prosthesis of claim 1, wherein the surfaces of the plates of the trial device have an abrasive surface for smoothing the vertebral end plates.

18. The abrasive trial device according to claims 17, wherein the abrasive surfaces comprise a series of cutting edges or a diamond studded surface, to produce a file or fine rasp-like surface.

19. The abrasive trial device according to claim 17, which can be connected to an insertion tool via the one or more lugs.

20. The abrasive trial device according to claim 17, which is formed as a single unit with an elongated handle enabling its insertion into an intervertebral space.

21. A sizing trial device having substantially the same dimensions as the intervertebral prosthesis of claim 1, and comprising one or more prism shaped lugs that correspond in size, shape and position to those of the intervertebral prosthesis according to claim 12.

22. An insertion tool comprising:
a shaft having a proximal end and a distal end wherein the proximal end comprises a grip and the distal end comprises two prosthesis engaging arms that are biased apart; and
a cylinder having a proximal and distal end in rotational engagement around the main shaft,
wherein the distal end of the cylinder contacts the prosthesis engaging arms, and wherein on rotation of the cylinder in a distal direction, the distal end of the cylinder forces the prosthesis engaging arms together to grip an intervertebral prosthesis according to claim 1.

23. A kit comprising one or more intervertebral prostheses according to claim 1, one or more abrasive trial devices according to any one of claim 17 and one or more sizing trial devices according to claim 21.

24. The kit according to claim 23 which additionally comprises the insertion tool according to claim 22.

25. The kit according to claim 23, which comprises a series of different sized intervertebral prostheses as well as a corresponding series of different sized abrasive trial devices and sizing trial devices.

26. A method for inserting the intervertebral prosthesis according to claim 1 comprising:
preparing the vertebral end plates between which the intervertebral prosthesis is to be inserted, by inserting and moving the abrasive trial device according to claim 17 within the intervertebral space so as to smooth the end plates and to ensure that the one or more lugs of the trial device engage a non-interstitial surface of the vertebrae; and
inserting the intervertebral prosthesis into the prepared intervertebral space; and
moving the prosthesis within the intervertebral space to ensure that the macro-textured surface engages the vertebral end plates.

27. A method for removing the intervertebral prosthesis according to claim 1 from an intervertebral space comprising passing a fine chisel or osteotome between the macro-textured surface of each end plate and the vertebral end plates.

28. The intervertebral prosthesis of claim 1 for use in therapy.

29. The abrasive trial device according to claim 17 or the sizing trial device according to claim 21 for use in therapy.
